(19) United States
(12) Patent Application Publication
(10) Pub. No.: US 2013/0090484 A1
(43) Pub. Date: Apr. 11, 2013

(54) PROCESS FOR MAKING AN INTERMEDIATE OF CABAZITAXEL

(71) Applicants: TsungYu Hsiao, Kaohsiung City (TW); HsinChang Tseng, Tainan City (TW)

(72) Inventors: TsungYu Hsiao, Kaohsiung City (TW); HsinChang Tseng, Tainan City (TW)

(73) Assignee: ScinoPharm Taiwan Ltd.

(21) Appl. No.: 13/271,192

(22) Filed: Oct. 11, 2011

(51) Int. Cl. C07D 305/14

(52) U.S. Cl. 549/510

(57) ABSTRACT

A novel process of making 7,10-dialkyl-10-DAB of formula (I)

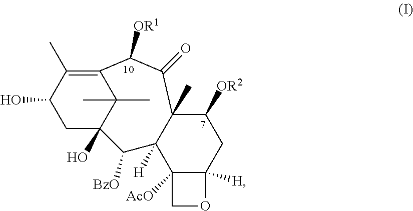

which is useful as a key intermediate for the preparation of cabazitaxel, comprises selective elaboration of positions 7 and 10 of 10-deacetylbaccatin III.

### CROSS-REFERENCES TO RELATED APPLICATIONS

NOT APPLICABLE

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The present invention relates to processes of making cabazitaxel and an intermediate thereof. Jevtana® is an injectable antineoplastic medicine whose active pharmaceutical ingredient (API), cabazitaxel, belongs to the taxane class, and is closely related in both chemical structure and mode of action to the anticancer drugs paclitaxel and docetaxel. Cabazitaxel is prepared by semi-synthesis from 10-deacetylbaccatin III (10-DAB) that is extracted from yew tree needles. The chemical name of cabazitaxel is (2α,5β,7β,10β,13α)-4-acetoxy-13-({(2R,3S)-3-[(tert-butoxycarbonyl)amino]-2-hydroxy-3-phenylpropanoyl}oxy)-1-hydroxy-7,10-dimethoxy-9-oxo-5,20-epoxy-tax-11-en-2-yl benzoate, which is marketed as a 1:1 acetone solvate (propan-2-one; refer to Formula A).

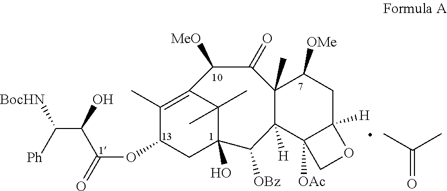

The acetone solvate of cabazitaxel is a white to off-white powder with a molecular formula of C₄₅H₅₇NO₁₄.C₃H₆O and a molecular weight of 894.01 grams/mole (for the acetone solvate), or 835.93 grams/mole for the solvent-free form.

Cabazitaxel is a dimethyl derivative of docetaxel, (also called dimethoxy docetaxel) which itself is semi-synthetic, and was originally developed by Rhone-Poulenc Rorer and was approved by the U.S. Food and Drug Administration (FDA) for the treatment of hormone-refractory prostate cancer. Cabazitaxel is a microtubule inhibitor.

Bouchard et al., in U.S. Pat. No. 5,847,170, describe cabazitaxel and its preparation methods. The entire content of this patent is incorporated herein by reference. One of the methods described in U.S. Pat. No. 5,847,170 is step-wise methylation of 10-deacetylbaccatin III (10-DAB) to provide key intermediate 4α-acetoxy-2α-benzoyloxy-5β,20-epoxy-1β,13α-dihydroxy-7β,10β-dimethoxy-9-oxo-11-taxene (7,10-dimethyl-10-DAB). The intermediate 7,10-dimethyl-10-DAB is then coupled with the protected side chain, and the oxazolidine protecting group is then removed from the side chain to give cabazitaxel. The step-wise methylation process disclosed in U.S. Pat. No. 5,847,170 is shown in FIG. 1.

Nonetheless, there are several disadvantages of the step-wise methylation process:

1) The protection of the hydroxyl group at position 13 is needed which is not economical, since an additional molar equivalent of silylating reagent and an additional molar equivalent of desilylating agent are then required.

2) The yield for the modification at position 10 with methyl iodide using sodium hydride to give the corresponding 10-methyl-7,13-diTES-10-DAB is low.

3) The yield for the removal of both silyl protecting groups of 10-methyl-7,13-diTES-10-DAB with hydrogen fluoride/triethylamine (3HF.NEt₃) to give 10-methyl-10-DAB is low.

Another method described in U.S. Pat. No. 5,847,170 is the bis-MTM ether route as shown in FIG. 2. However, 7,10-bis-MTM derivatives of 10-DAB are not directly accessible from 10-DAB itself when they are formed using Ac₂O/DMSO (Pummerer reaction) because these conditions lead to concomitant oxidation of the hydroxyl group at position 13 to the corresponding ketone. Furthermore, the dimethylthiomethylation of hydroxyl groups at positions 7 and 10 is slow and proceeds in low yield.

Therefore, there is a need for the development of improved processes for the preparation of cabazitaxel and its key intermediate, 7,10-dimethyl-10-DAB.

### BRIEF SUMMARY OF THE INVENTION

The present invention provides a process for making 7,10-dialkyl-10-DAB compounds of formula (I), which are themselves useful materials for the synthesis of cabazitaxel.

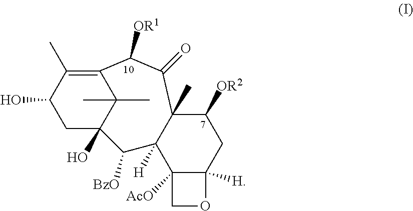

In some embodiments of the invention, the process includes selective protection of the C7-hydroxyl group of 10-DAB with silyl ether groups, followed by alkylation of the C10-hydroxyl group and conversion to the 7,10-dialkyl-10-DAB. In some embodiments, the 7,10-dialkyl-10-DAB is further elaborated to provide cabazitaxel.

### BRIEF DESCRIPTION OF THE DRAWINGS

FIG. 1 shows the step-wise methylation of 10-DAB disclosed in U.S. Pat. No. 5,847,170.

FIG. 2 shows the synthesis of cabazitaxel via a Bis-MTM ether route disclosed in U.S. Pat. No. 5,847,170.

FIG. 3 shows the selective protection of the C7 hydroxyl group of 10-DAB using the methods of the invention.

FIG. 4 shows the synthesis of 7,10-dialkyl-10-DAB using the methods of the invention.

### DETAILED DESCRIPTION OF THE INVENTION

### I. General

The present invention is based on the unexpected discovery that the C7 hydroxyl group of 10-DAB can be selectively protected without prior protection of the C10 and C13 hydroxyl groups. Accordingly, the invention provides mild and atom-economical methods for the production of 7,10-dialkyl-10-DAB which can be used to synthesize cabazitaxel. The methods can be conducted with a variety of silylation agents, generally using low-temperature conditions.

### II. Definitions

The term “alkyl”, by itself or as part of another substituent, means, unless otherwise stated, a straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like.

As used herein, the terms “halide,” “halo,” or “halogen,” by themselves or as part of another substituent, mean a fluorine, chlorine, bromine, or iodine atom.

As used herein, the terms “aryl” and “aromatic ring” refer to a polyunsaturated, hydrocarbon group which can be a single ring or multiple rings (up to three rings) which are fused together or linked covalently. Non-limiting examples of aryl groups include phenyl, naphthyl and biphenyl.

As used herein, the term “contacting” refers to the process of bringing into contact at least two distinct species such that they can react. It should be appreciated, however, the resulting reaction product can be produced directly from a reaction between the added reagents or from an intermediate from one or more of the added reagents which can be produced in the reaction mixture.

As used herein, the terms “selective” and “selectively” refer to methods that provide a product, the majority of which is a single chemical species. The product may be obtained, for example, by converting a certain functional group within a molecule to a new moiety while leaving other function groups within the molecule substantially unchanged. Such methods may employ orthogonal protecting group strategies to address particular functional groups, or they may rely on the intrinsic chemical properties of a given functional group to direct desired reactivity.

### III. Embodiments of the Invention

Some embodiments of the present invention provide a process for making 7,10-dialkyl-10-DAB of formula (I):

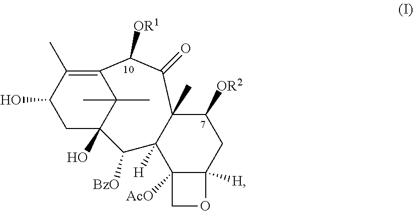

wherein each of R¹ and R², which may be identical or different, is an unbranched or a branched C₁-C₆ alkyl chain. The process includes:
(a) contacting 10-DAB of formula (II):

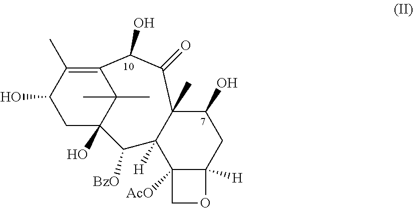

with a compound of formula (VII):

(R″)₃—Si-Hal (VII)

to selectively obtain a compound of formula (III):

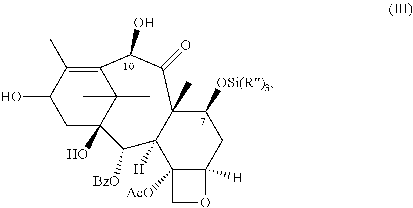

wherein each R″ is selected from an unbranched or a branched C₁-C₆ alkyl chain and C₆-C₁₀ aromatic rings; and Hal is halide. In some embodiments, the compound of formula VII is triethylsilylchloride.

In some embodiments, the process is conducted at not more than 0° C., or at from 0° C. to −20° C., or at from about −10° C. to about −20° C.

In some embodiments, the process is carried out in the presence of an organic solvent, such as dimethylformamide (DMF) or THF, with a weak base, such as pyridine, a tertiary amine, 1,8-diazabicyclo[5.4.0]undec-7-ene, 1,5-diazabicyclo[4.3.0]non-5-ene, a saturated heterocyclic base, a pyridine derivative or an aromatic heterocyclic base. In some embodiments, the weak base is imidazole.

In some embodiments, the process includes:

(b) contacting a compound of formula (III) with an alkyl halide, a dialkyl sulfate, a trialkyl oxonium salt, or an alkyl sulfonate in the presence of a base to obtain a compound of formula (IV):

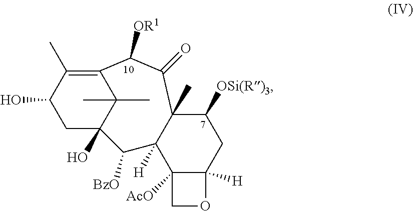

wherein R¹ and R″ are defined as above;
(c) contacting the compound of formula (IV) with a desilylation agent to obtain a compound of formula (V):

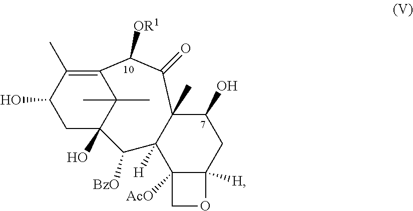

wherein R¹ is defined as above; and
(d) contacting the compound of formula (V) with an alkyl halide, a dialkyl sulfate, a trialkyl oxonium salt, or an alkyl sulfonate in the presence of a base to obtain the product of formula (I), wherein R¹, R² and R″ are defined as above.

The synthetic steps described above can be carried out in an organic solvent, such as THF or any other suitable solvent. In some embodiments, the alkylation of the C10-hydroxyl group is first conducted at low temperature, preferably at not more than −20° C., and then warmed to room temperature. In some embodiments, the base used for the alkylation of the C10-hydroxyl group may be any suitable base, preferably a strong base. Examples of strong bases include, but are not limited to, an alkali metal hydride such as sodium hydride (NaH), potassium hydride (KH), lithium hydride (LiH), calcium hydride (CaH₂), or magnesium hydride (MgH₂); an alkali metal alkoxide; a mixture of an alkali metal amide, such as lithium bis(trimethylsilyl)amide (LiHMDS), sodium bis(trimethylsilyl)amide (NaHMDS), potassium diisopropylamide (KDA), or lithium diisopropylamide (LDA); an alkali metal tert-butoxide; or a mixture of an alkyllithium and an alkali metal tert-butoxide. In some embodiments, the base is LiHMDS.

In some embodiments, the desilylation agent used for deprotection of the C7-hydroxyl group is tetrabutylammonium fluoride (TBAF), hydrofluoric acid, caesium fluoride, potassium fluoride, or a strong acid, such as hydrochloric acid, toluenesulfonic acid or trifluoroacetic acid.

The base used for alkylation of the C7-hydroxyl group may be any suitable base. In some embodiments, the base used for alkylation of the C7-hydroxyl group is a strong base. Strong bases include, but are not limited to, an alkali metal hydride, such as sodium hydride (NaH), potassium hydride (KH), lithium hydride (LiH), calcium hydride (CaH₂), or magnesium hydride (MgH₂); an alkali metal alkoxide; a silver oxide; a mixture of an alkali metal amide, such as lithium bis(trimethylsilyl)amide (LiHMDS), sodium bis(trimethylsilyl)amide (NaHMDS), potassium diisopropylamide (KDA), or lithium diisopropylamide (LDA); an alkali metal tert-butoxide; or a mixture of an alkyllithium and an alkali metal tert-butoxide.

The alkylation of the C7- and C10-hydroxyl groups is conducted with any suitable alkylating agent including, but not limited to, an alkyl halide, a dialkyl sulfate, a trialkyl oxonium salt or an alkyl sulfonate, preferably an alkyl halide, such as methyl iodide.

In some embodiments, each of R¹ and R² in formula (I) can be an unbranched or a branched C₁-C₃ alkyl chain which may be identical or different. In some embodiments, each of R¹ and R² is a methyl group. In some embodiments, the process includes converting the compound of formula I, wherein R¹ and R² are methyl groups, to cabazitaxel.

As described above, the present invention discloses a method for the preparation of 7,10-dialkyl-10-DAB, which may be elaborated to yield cabazitaxel. In accordance with an embodiment of the present invention, the preparation method may comprise selective protection of 10-DAB via silylation of the hydroxyl group at position 7 at between 0° C. to −20° C.

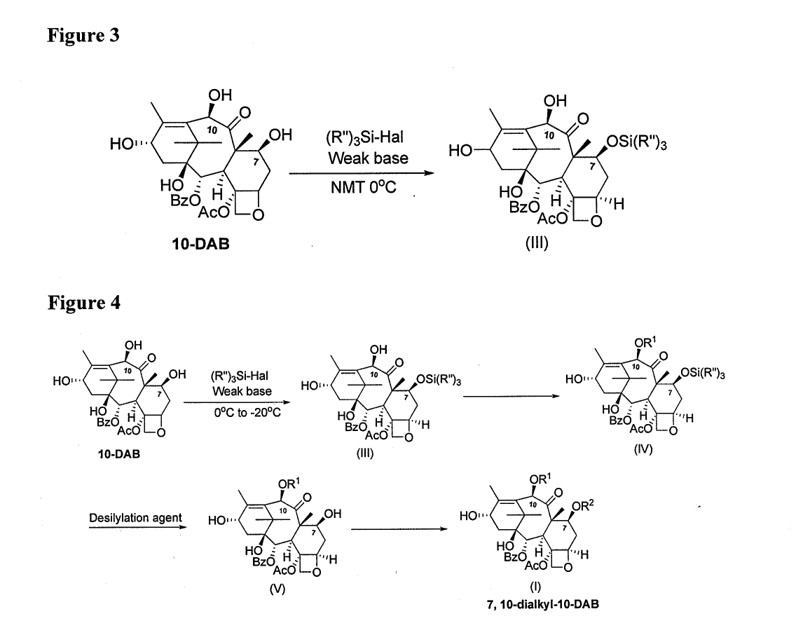

An embodiment of the process is shown in FIG. 3. In formula (III), R″ is an unbranched or a branched C₁-C₆ alkyl chain or a C₆-C₁₀ aromatic ring, preferably an unbranched or a branched C₁-C₆ alkyl chain such as ethyl, and Hal is halide, such as chloride.

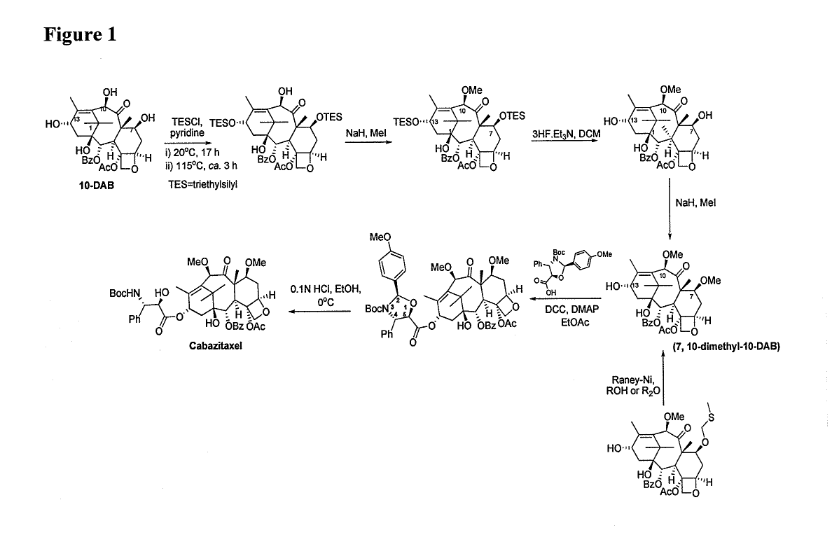

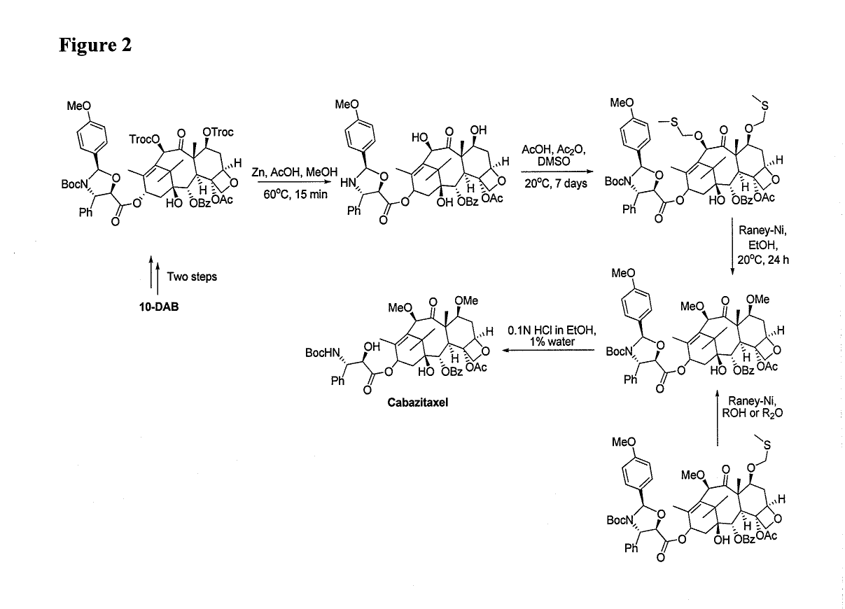

The aforementioned process further includes selective alkylation at position 10 followed by desilylation and further alkylation at position 7 to obtain 7,10-dialkyl-10-DAB. This 7,10-dialkyl-10-DAB can be further converted to cabazitaxel as shown in FIG. 1 and FIG. 2.

An embodiment of the overall process is summarized in FIG. 4. In FIG. 4, R″ and Hal are defined as above. Each of R¹ and R², which may be identical or different, is independently an unbranched or a branched C₁-C₆ alkyl chain. Preferably each of R¹ and R², which may be identical or different, is independently an unbranched or a branched C₁-C₃ alkyl chain. More preferably each of R¹ and R² is a methyl group.

In comparison with the prior art, the present invention has the following advantages:

1) The reaction of a 10-DAB compound of formula (I) with (R″)₃—Si-Hal is carried out under milder conditions, preferably at not more than 0° C. In comparison, the silylation of hydroxyl groups at positions 7 and 13, as disclosed in U.S. Pat. No. 5,847,170, is conducted at 20° C. for 17 hours and then heated to about 115° C. for about 3 hours, which is less efficient from an industrial perspective.
2) The inventors of the present invention unexpectedly discovered that only one silyl group is required to protect 10-DAB when a lower temperature is used, e.g. not more than 0° C. Therefore, the present invention is more atom economical because only one molar equivalent of silylating reagent and one molar equivalent of desilylating agent are required. In comparison, U.S. Pat. No. 5,847,170 discloses the method that requires two molar equivalents of silylating reagent and desilylating agent.
3) In accordance with the present invention, the yield for the removal of the silyl protecting group from the 7-position of a compound of formula (IV) is more than 80%. In comparison, the yield of the removal of both silyl protecting groups of 10-methyl-7,13-diTES-10-DAB, as disclosed in U.S. Pat. No. 5,847,170, is around 70%.
4) In accordance with the present invention, the overall yield for the synthesis of 7,10-dialkyl-10-DAB is around 40%. In comparison, the step-wise methylation method taught in U.S. Pat. No. 5,847,170 is less than 20%.

### EXAMPLES

The following examples are provided for the purpose of further illustration only and are not intended to be limitations on the disclosed invention.

### Example 1

### Preparation of 7-(triethylsilyl)-10-deacetyl baccatin III

Chlorotriethylsilane (3.7 g) was slowly added to a chilled mixture of 10-deacetyl baccatin III (8.0 g) and imidazole (3.1 g) in dimethylformamide (DMF). After stirring at 0° C. to −20° C. until the reaction was completed, the product mixture was slowly added to a mixture of water and toluene and stirred. n-Hexane was added to the slurry and the mixture was stirred. The product was filtered and the wet cake was dissolved in EtOAc. The solution was washed with saturated sodium chloride solution, and the EtOAc layer was separated and concentrated under reduced pressure until most of the EtOAc was removed. n-Heptane was added and replacement distillation was carried out under reduced pressure until most of the EtOAc and n-heptane mixture was removed. n-Heptane was added, stirred, and 7-(triethylsilyl)-10-deacetyl baccatin III was filtered and dried under vacuum at not less than 40° C. to provide 7-(Triethylsilyl)-10-deacetyl baccatin III (95% yield).

¹H NMR (400 Hz, MHz, CDCl₃) δ 8.13 (d, J=8.0 Hz, 2H), 7.61 (m, 1H), 7.48 (m, 2H), 5.62 (d, J=7.2 Hz, 1H), 5.19 (s, 1H), 4.97 (dd, J=13.2, 1.6 Hz, 1H), 4.88 (m, 1H), 4.43 (dd, J=10.8, 6.8 Hz, 1H), 4.32 (dd, J=86, 8.8 Hz, 2H), 4.32 (m, 1H), 3.97 (d, J=7.2 Hz, 1H), 2.53-2.45 (m, 1H), 2.30 (s, 3H), 2.29-2.27 (m, 2H), 2.13 (s, 3H), 195-1.88 (m, 1H), 1.76 (s, 3H), 1.60 (m, 1H), 1.1 (m, 6H), 0.98-0.93 (m, 9H), 0.63-0.55 (m, 6H)

### Example 2

### Preparation of 10-deacetyl-10-methyl-7-triethylsilyl baccatin III

A solution of 7-(triethylsilyl)-10-deacetyl baccatin III (21.6 g) was prepared in THF. Then lithium bis(trimethylsilyl)amide (LiHMDS) in THF was added to the solution at not more than −20° C. After stirring, methyl iodide was added dropwise. The mixture was warmed to 0° C. over 1 hour and was then warmed to room temperature. The reaction was quenched with saturated NH₄Cl and extracted with THF. The organic layer was concentrated, and THF and n-heptane were added to cause precipitation. The solid was collected and dried under vacuum at not more than 50° C. to provide 10-deacetyl-10-methyl-7-triethylsilyl baccatin III (82% yield).

¹H NMR (400 Hz, MHz, CDCl₃) δ 8.13 (d, J=8.0, 2H), 7.62 (t, J=7.2, 1H), 7.49 (t, J=7.6 Hz, 2H), 5.62 (d, J=6.8 Hz, 1H), 4.98-4.97 (m, 1H), 4.96 (s, 1H), 4.97-4.93 (m, 1H), 4.45 (m, 1H), 4.24 (dd, J=60, 8.4 Hz, 2H), 3.90 (d, J=7.2 Hz, 1H), 3.43 (s, 3H), 2.52-2.47 (m, 1H), 2.31 (s, 3H), 2.31-2.28 (m, 1H), 2.13 (s, 3H), 2.16-2.13 (m, 1H), 1.94-1.89 (m, 1H), 1.70 (s, 3H), 1.19 (s, 3H), 1.09 (s, 3H), 0.90 (m, 6H), 0.88 (m, 6H), 0.63-0.55 (m, 5H).

### Example 3

### Preparation of 10-deacetyl-10-methyl baccatin III

A solution of 10-deacetyl-10-methyl-7-triethylsilyl baccatin III (40.3 g) in THF and 1M tetrabutylammonium fluoride (TBAF) in THF was stirred at room temperature. Water was added to the reaction mixture, and the mixture was then concentrated to provide a solid which was filtered and washed with methyl tert-butyl ether (MTBE). The crude solid was dissolved in THF and was precipitated by the addition of water. The solid was filtered and dried under vacuum at not less than 55° C. to provide 10-deacetyl-10-methyl baccatin III (83% yield).

¹H NMR (400 Hz, MHz, DMSO) δ 8.02 (dd, J=8.4, 6.8 Hz, 2H), 7.68-7.64 (m, 1H), 7.57 (t, J=7.6 Hz, 2H), 5.39 (d, J=6.8 Hz, 1H), 5.28 (m, 1H), 5.01 (m, 1H), 4.92 (d, J=8.0 Hz, 1H) 4.89 (s, 1H), 4.68-4.64 (m, 1H), 4.15-4.11 (m, 1H), 4.02 (s, 2H), 3.75 (d, J=6.8 Hz, 1H), 3.31 (s, 3H), 2.52-2.50 (m, 2H), 2.23-2.22 (m, 1H), 2.19-2.16 (m, 4H), 2.19 (s, 3H), 1.65-1.63 (m, 1H), 1.48 (s, 3H), 0.95-0.92 (m, 6H).

### Example 4

### Preparation of 7,10-dimethyl-10-DAB

A suspension of 10-deacetyl-10-methyl baccatin III (20 g) in a solution of MeI in THF was added dropwise to a prewashed suspension of potassium hydride in THF at 0° C. The mixture was allowed to warm to room temperature, and after stirring the reaction mixture was poured into a mixture of diisopropyl ether and water. The mixture was filtered through a sintered funnel to provide 7,10-dimethyl-10-DAB, which was dried under vacuum at 50° C. (61% yield).

¹H NMR (400 Hz, MHz, DMSO) δ 8.02 (d, J=7.2 Hz, 2H), 7.68-7.65 (m, 1H), 7.57 (t, J=8 Hz, 2H), 5.39 (d, J=6.8 Hz, 1H), 5.31 (d, J=4.4 Hz, 1H), 4.98 (d, J=9.2 Hz, 1H) 4.75 (s, 1H), 4.66-4.65 (m, 1H), 4.40 (s, 1H), 4.06-4.01 (m, 2H), 3.83-3.79 (m, 1H), 3.75 (d, J=7.2 Hz, 1H), 3.30 (s, 3H), 3.22 (s, 3H), 2.69-2.65 (m, 1H), 2.21 (s, 3H), 2.20-2.17 (m, 2H), 1.98 (s, 3H), 1.52 (s, 3H), 1.52-1.46 (m, 1H), 0.91 (s, 6H).

### Example 5

### Preparation of 4-α-acetoxy-2α-benzoyloxy-5β,20-epoxy-1β-hydroxy-7β,10β-dimethoxy-9-oxo-11-taxen-13α-yl(2R,4S,5R)-3-tert-butoxycarbonyl-2-(4-methoxyphenyl)-4-phenyl-1,3-oxazolidine-5-carboxylate

7,10-dimethyl-10-DAB (200 mg), 4-dimethylaminopyridine (4-DMAP), and (2R,4S,5R)-3-tert-butoxycarbonyl-2-(4-methoxyphenyl)-4-phenyl-1,3-oxazolidine-5-carboxylic acid (280 mg) were dissolved in THF. Dicyclohexylcarbodiimide was then added to the mixture. After the reaction was completed, the reaction mixture was quenched with HCl. The reaction mixture was filtered with filter paper and washed with EtOAc. The filtrate was washed with NaHCO₃ followed by water.

The organic layer was reduced under vacuum to provide an oil that was purified by column chromatography with EtOAc/n-heptane to furnish 4-α-acetoxy-2α-benzoyloxy-5β,20-epoxy-1β-hydroxy-7β,10β-dimethoxy-9-oxo-11-taxen-13α-yl(2R,4S,5R)-3-tert-butoxycarbonyl-2-(4-methoxyphenyl)-4-phenyl-1,3-oxazolidine-5-carboxylate as a white amorphous solid.

¹H NMR (400 Hz, MHz, CDCl₃) δ 8.04 (dd, J=8, 1.2 Hz, 2H), 7.65-7.61 (m, 1H), 7.52-7.44 (m, 9H), 6.93 (dd, J=6.8, 2.8 Hz, 2H), 6.40-6.39 (m, 1H), 6.16 (m, 1H), 5.60 (d, J=7.2 Hz, 1H), 5.44 (m, 1H), 4.91 (d, J=8.4 Hz, 1H), 4.72 (s, 1H), 4.59 (d, J=5.2 Hz, 1H), 4.22 (dd, J=46, 8.4 Hz, 2H), 3.85-3.80 (m, 4H), 3.74 (d, J=6.8 Hz, 1H), 3.42 (s, 3H), 3.29 (s, 3H), 2.70-2.63 (m, 1H), 2.11-2.05 (m, 2H), 1.83 (s, 3H), 1.78-1.59 (m, 2H), 1.63 (s, 3H), 1.59 (s, 3H), 1.22 (s, 3H), 1.18 (s, 3H), 1.07 (s, 9H).

¹³C NMR (100 Hz, MHz, CDCl₃) δ 204.8, 169.9, 169.5, 166.9, 160.4, 151.5, 139.0, 135.1, 133.7, 130.1, 129.3, 129.0, 128.7, 128.6, 128.2, 126.6, 113.9, 92.6, 84.1, 82.4, 81.3, 80.9, 80.6, 79.1, 77.3, 74.7, 71.8, 63.7, 57.1, 56.7, 55.3, 47.3, 43.2, 35.4, 34.0, 31.9, 27.8, 26.7, 25.6, 24.9, 21.6, 20.9, 13.9, 10.3.

### Example 6

### Preparation of Cabazitaxel

A 2-Methyl-THF solution of 4-α-acetoxy-2α-benzoyloxy-5β,20-epoxy-1,3-hydroxy-7β, 10β-dimethoxy-9-oxo-11-taxen-13α-yl(2R,4S,5R)-3-tert-butoxycarbonyl-2-(4-methoxyphenyl)-4-phenyl-1,3-oxazolidine-5-carboxylate (1.0 g) and hydrochloric acid/MeOH was stirred at room temperature. After the reaction was completed, the mixture was diluted with EtOAc and quenched with NaHCO₃. The organic phase was removed in vacuo to provide an oil that was precipitated with EtOAc/n-heptane to afford cabazitaxel (about 83% yield).

¹H NMR (400 Hz, MHz, CDCl₃) δ 8.04 (dd, J=8, 1.2 Hz, 2H), 7.63-7.59 (m, 1H), 7.51-7.47 (m, 2H), 7.40-7.39 (m, 4H), 7.34-7.28 (m, 1H), 6.24-6.20 (m, 1H), 5.63 (d, J=7.2 Hz, 1H), 5.51 (m, 1H), 5.29-5.26 (m, 1H), 4.98 (d, J=8.4 Hz, 1H), 4.81 (s, 1H), 4.63 (m, 1H), 4.23 (dd, J=41, 8.4 Hz, 2H), 3.88-3.84 (m, 1H), 3.82 (d, J=6.8 Hz, 1H), 3.58 (m, 1H), 4.46 (s, 3H), 3.31 (s, 3H), 2.72-2.68 (m, 1H), 2.37 (s, 3H), 2.30-2.27 (m, 2H), 1.89 (s, 3H), 1.89-1.76 (m, 2H), 1.72 (s, 3H), 1.37 (s, 9H), 1.22 (s, 3H), 1.21 (s, 3H).

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A process of making 7,10-dialkyl-10-DAB of formula (I): 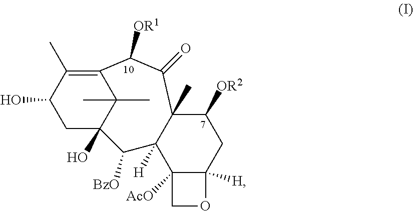
wherein each of R¹ and R², which maybe identical or different, is unbranched or branched C₁-C₆ alkyl, said process comprising:
(a) contacting a compound of formula (II): 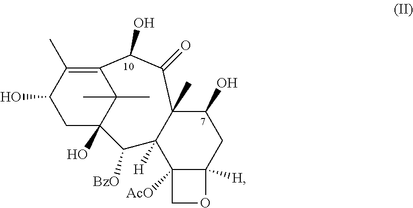
with a compound of formula (VII):
(R″)₃—Si-Hal (VII)
to selectively obtain a compound of formula (III): 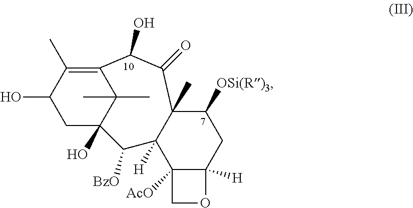
wherein each R″ is independently selected from the group consisting of branched or unbranched C₁-C₆ alkyl and C₆-C₁₀ aryl, and Hal is halide.

2. The process according to claim 1, wherein the reaction is conducted at between 0° C. to about −20° C.

3. The process according to claim 1, wherein the reaction is conducted at about −10° C. to about −20° C.

4. The process according to claim 1, wherein the reaction is conducted in the presence of a weak base.

5. The process according to claim 4, wherein said weak base is selected from pyridine, a tertiary amine, 1,8-diazabicyclo[5.4.0]undec-7-ene, 1,5-diazabicyclo[4.3.0]non-5-ene, a saturated heterocyclic base, a pyridine derivative and an aromatic heterocyclic base.

6. The process according to claim 1, wherein said compound of formula (VII) is triethylsilylchloride.

7. The process according to claim 1, wherein each of R¹ and R², which may be identical or different, is unbranched or branched C₁-C₃ alkyl.

8. The process according to claim 1, wherein each of R¹ and R² is methyl.

9. The process according to claim 1 further comprising:
(b) contacting a compound of formula (III) with an alkyl halide, a dialkyl sulfate, a trialkyl oxonium salt, or an alkyl sulfonate in the presence of a base to obtain a compound of formula (IV): 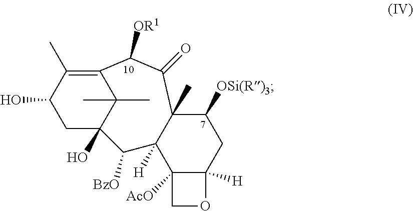
(c) contacting a compound of formula (IV) with a desilylation agent to obtain a compound of formula (V): 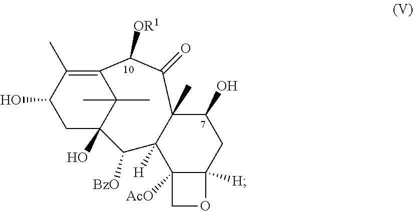
(d) contacting a compound of formula (V) with an alkyl halide, a dialkyl sulfate, a trialkyl oxonium salt, or an alkyl sulfonate in the presence of a base to obtain the product of formula (I), wherein R¹, R² and R″ are defined as in claim 1.

10. The process according to claim 9, wherein the base of the step (b) is a strong base selected from an alkali metal hydride, an alkali metal alkoxide, a mixture of an alkali metal amide, an alkali metal tert-butoxide, and a mixture of an alkyllithium and an alkali metal tert-butoxide.

11. The process according to claim 9, wherein the base of the step (d) is a strong base selected from an alkali metal hydride, an alkali metal alkoxide, silver oxide, a mixture of an alkali metal amide, an alkali metal tert-butoxide, and a mixture of an alkyllithium and an alkali metal tert-butoxide.

12. The process according to claim 9, wherein the desilylation agent is selected from tetrabutylammonium fluoride, hydrofluoric acid, caesium fluoride, potassium fluoride and strong acid.

13. The process according to claim 1 further comprising converting the compound of formula (I) into cabazitaxel, wherein each of R¹ and R² is methyl.
